SEQUENCE LISTING (8 sequences)

SEQ ID NO: 1 - DNA, 27 bp, Artificial Sequence
  Mutagenic primer
tatttatgcagtaaattgtggattaac

SEQ ID NO: 2 - DNA, 25 bp, Artificial Sequence
  Mutagenic primer
gagaattcttaattacaaccttcga

SEQ ID NO: 3 - DNA, 32 bp, Artificial Sequence
  Mutagenic primer
ctcgaattcttaaataggtctatcttgttccg

SEQ ID NO: 4 - DNA, 32 bp, Artificial Sequence
  Mutagenic primer
ctcgaattcttaagaataagaggttggagaag

SEQ ID NO: 5 - DNA, 31 bp, Artificial Sequence
  Mutagenic primer
gggaattcttatgggtcaaaagttttttgag

SEQ ID NO: 6 - DNA, 25 bp, Artificial Sequence
  Mutagenic primer
aggaattcttatggattaacatttg

SEQ ID NO: 7 - DNA, 26 bp, Artificial Sequence
  Mutagenic primer
aggaattcttaaaattgtggattaac

SEQ ID NO: 8 - DNA, 38 bp, Artificial Sequence
  Mutagenic primer
aggaattcttattaagtaaattgtggattaacatttgg

(19) United States
(12) Patent Application Publication
(10) Pub. No.: US 2006/0166236 A1
(43) Pub. Date: Jul. 27, 2006

(54) Allosteric enzyme coupled immunoassay (AECIA)

(76) Inventor: Chong-Sheng Yuan, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/304,010

(22) Filed: Dec. 15, 2005

Related U.S. Application Data
(60) Provisional application No. 60/636,472, filed on Dec. 15, 2004.

(51) Int. Cl. C12Q 1/68; G01N 33/566

(52) U.S. Cl. 435/6; 436/501; 436/90

(57) ABSTRACT

The present invention is directed to methods and compositions for an allosteric enzyme coupled assay, and preferably to an allosteric enzyme coupled immunoassay (AECIA). The assay uses an allosteric enzyme to generate a readout signal. The assay is based on the competition between an analyte in a sample and an analyte or analyte analog conjugated to an allosteric regulator with a specific binding reagent for the analyte. In the absence of any analyte in the sample, an analyte or an analyte analog conjugated to an allosteric regulator binds to the specific binding reagent and such binding prevents or reduces the allosteric regulator's regulation, e.g., activation, on the allosteric enzyme. An analyte, if present in the sample, competes with the analyte or analyte analog conjugated to the allosteric regulator for binding with the specific binding reagent, reduces or prevents binding of the specific binding reagent to the analyte or analyte analog conjugated to the allosteric regulator, leading to increased regulation, e.g., activation, of the enzyme. 1-substituted-β-D-fructofuranose 2,6-bisphosphate compounds and conjugates comprising the same are provided. Kits comprising the conjugates, and methods using the conjugates for assaying an analyte are further provided.

### CROSS-REFERENCES TO RELATED APPLICATIONS

This application claims the priority benefit of U.S. provisional patent application Ser. No. 60/636,472, filed Dec. 15, 2004, the content of which is incorporated by reference in its entirety.

### FIELD OF THE INVENTION

The present invention relates to methods and kits of assaying an analyte in a sample using allosteric enzyme coupled assays. The present invention also relates to 1-substituted-β-D-fructofuranose 2,6-bisphosphate compounds, the conjugates comprising the compounds, kits comprising the conjugate, and methods using the conjugate for assaying an analyte.

### BACKGROUND OF THE INVENTION

U.S. Pat. No. 4,134,792 is directed to a specific binding assay method employing, as a labeling substance, a reversibly binding enzyme modulator for the detection of a ligand in a liquid medium. The method follows conventional specific binding assay techniques of either the homogeneous or heterogeneous type wherein the liquid medium to be assayed is combined with reagent means that includes a labeled conjugate to form a binding reaction system having a bound-species and a free-species of the conjugate. The amount of conjugate resulting in the bound-species or the free-species is a function of the amount of ligand present in the liquid medium assayed. According to U.S. Pat. No. 4,134,792, the labeled conjugate comprises a reversibly binding enzyme modulator covalently linked to a binding component of the binding reaction system. The distribution of the conjugate between the bound-species and the free-species is determined by addition of an enzyme whose activity is affected, either decreased or increased, by said modulator and measuring the resulting activity of the enzyme. The enzyme modulator may be a conventional enzyme inhibitor, preferably of the coompetitive type, or an allosteric effector.

However, allosteric enzyme coupled assay, e.g., allosteric enzyme coupled immunoassay (AECIA), using an optimized pair of an allosteric enzyme and its allosteric effector(s) is needed to achieve a desired assay sensitivity range. The present invention addresses this and other related needs in the art.

### SUMMARY OF THE INVENTION

The present invention is directed to methods and compositions for an allosteric enzyme coupled assay, and preferably to an allosteric enzyme coupled immunoassay (AECIA). The assay uses an allosteric enzyme to generate a readout signal. The assay is based on the competition between an analyte in a sample and an analyte or analyte analog conjugated to an allosteric regulator with a specific binding reagent for the analyte. In the absence of any analyte in the sample, an analyte or an analyte analog conjugated to an allosteric regulator binds to the specific binding reagent and such binding prevents or reduces the allosteric regulator's regulation, e.g., activation, on the allosteric enzyme. An analyte, if present in the sample, competes with the analyte or analyte analog conjugated to the allosteric regulator for binding with the specific binding reagent, reduces or prevents binding of the specific binding reagent to the analyte or analyte analog conjugated to the allosteric regulator, leading to increased regulation, e.g., activation, of the enzyme.

In one aspect, a method for assaying an analyte in a sample is provided, which method comprises: a) contacting a sample containing an analyte or suspected of containing an analyte with a specific binding reagent for said analyte in the presence of an allosteric enzyme, e.g., an allosteric phosphofructokinase, and an analyte or analyte analog conjugated to an allosteric regulator of said enzyme, e.g., fructose-2,6-bisphosphate or fructose-1,6-bisphosphate or a compound comprising fructose-2,6-bisphosphate or fructose-1,6-bisphosphate, under conditions such that binding of said specific binding reagent to said analyte or analyte analog conjugated to said allosteric regulator prevents or reduces regulation, e.g., activation, of said enzyme by said allosteric regulator, and said analyte, if present in said sample, competes with said analyte or analyte analog conjugated to said allosteric regulator for binding with said specific binding reagent, reduces or prevents binding of said specific binding reagent to said analyte or analyte analog conjugated to said allosteric regulator, leading to increased regulation, e.g., activation, of said enzyme; and b) determining the presence, absence and/or amount of said analyte in said sample by assessing activity of said enzyme.

In another aspect, a kit for assaying an analyte in a sample is provided, which kit comprises: a) a specific binding reagent for an analyte; b) an allosteric enzyme, e.g., an allosteric phosphofructokinase; c) an analyte or analyte analog conjugated to an allosteric regulator of said enzyme, e.g., fructose-2,6-bisphosphate or fructose-1,6-bisphosphate or a compound comprising fructose-2,6-bisphosphate or fructose-1,6-bisphosphate; and d) means for assessing activity of said enzyme.

In another aspect, a 1-substituted-β-D-fructofuranose 2,6-bisphosphate compound, or a salt thereof, is provided, which compound, or a salt thereof, has the following formula I:

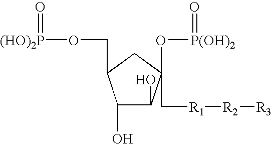

wherein R₁ is O, N or S; R₂ is a C₃ to C₃₀ alkyl group; and R₃ is OH, SH, NH₂, COOH, CONH₂, or COOR₄, wherein R₄ is a C₁ to C₃₀ alkyl group. An analyte, analyte analog or a specific binding partner for an analyte conjugated to the 1-substituted-β-D-fructofuranose 2,6-bisphosphate compound is also provided. Kits comprising the conjugate, and methods using the conjugate for assaying an analyte are further provided.

### BRIEF DESCRIPTION OF THE DRAWINGS

FIG. 1 illustrates an exemplary allosteric enzyme coupled immunoassay (AECIA), where AR stands for allosteric regulator and PFK stands for phosphofructokinase.

FIG. 2 illustrates enzymatic reaction scheme of a PFK based allosteric enzyme coupled immunoassay.

FIG. 3 illustrates activation of potato tuber PPi-PFK by Fructose 2,6 Pi.

FIG. 4 illustrates activation of rat liver PFK by Fructose 2,6 Pi.

FIG. 5 illustrates an exemplary synthesis scheme of 1-substituted-β-D-fructofuranose 2,6-bisphosphate (SFT-BP).

### DETAILED DESCRIPTION OF THE INVENTION

Definitions

Unless defined otherwise, all technical and scientific terms used herein have the same meaning as is commonly understood by one of ordinary skill in the art to which this invention belongs. All patents, patent applications (published or unpublished) and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “phosphofructokinase (PFK)” refers to an enzyme, or a functional fragment or derivative thereof, that converts fructose 6-phosphate to fructose 1,6-bisphosphate. The phosphofructokinase can be ATP or pyrophosphate dependent. It is intended to encompass phosphofructokinase with conservative amino acid substitutions that do not substantially alter its activity.

As used herein, “fructose-bisphosphate aldolase (FBA)” refers to an enzyme, or a functional fragment or derivative thereof, that converts D-fructose-1,6-bisphosphate to Glycerone phosphate and D-glyceraldehyde 3-phosphate. It is intended to encompass fructose-bisphosphate aldolase with conservative amino acid substitutions that do not substantially alter its activity.

As used herein, “glyceraldehyde-3-phosphate dehydrogenase (GPD)” refers to an enzyme, or a functional fragment or derivative thereof, that catalyses the reversible oxidative phosphorylation of glyceraldehyde-3-phosphate. It is intended to encompass glyceraldehyde-3-phosphate dehydrogenase with conservative amino acid substitutions that do not substantially alter its activity.

As used herein, “pyruvate kinase (PK)” refers to an enzyme, or a functional fragment or derivative thereof, that converts phospho(enol)pyruvate and ADP to pyruvate and ATP. It is intended to encompass pyruvate kinase with conservative amino acid substitutions that do not substantially alter its activity.

As used herein, “lactate dehydrogenase (LDH)” refers to an enzyme, or a functional fragment or derivative thereof, that catalyses the formation and removal of lactate according to the equation: pyruvate+NADH=lactate+NAD⁺. It is intended to encompass lactate dehydrogenase with conservative amino acid substitutions that do not substantially alter its activity.

As used herein, “triose phospphate isomerase (TPI)” refers to an enzyme, or a functional fragment or derivative thereof, that catalyses the reversible isomerization between D-glyceraldehyde-3-Pi and Dihydroxyacetone Pi. It is intended to encompass triose phospphate isomerase with conservative amino acid substitutions that do not substantially alter its activity.

As used herein, “glycerol-3-phosphate dehydrogenase (Glycerol-3PDH)” refers to an enzyme, or a functional fragment or derivative thereof, that catalyses the reaction between Dihydroxyacetone Pi and NADH to form Glycerol-3-Pi and NAD⁺. It is intended to encompass glycerol-3-phosphate dehydrogenase with conservative amino acid substitutions that do not substantially alter its activity.

As used herein, “binding of said specific binding reagent to said analyte or analyte analog conjugated to said fructose-2,6-bisphosphate or fructose-1,6-bisphosphate prevents or reduces regulation of said allosteric phosphofructokinase by said fructose-2,6-bisphosphate or fructose-1,6-bisphosphate” means that binding of the specific binding reagent to the analyte or analyte analog conjugate leads to at least 10% reduction of the regulation of the allosteric phosphofructokinase by fructose-2,6-bisphosphate or fructose-1,6-bisphosphate. Preferably, binding of the specific binding reagent to the analyte or analyte analog conjugate leads to 20%, 30%, 40%, 50%, 60%, 70%, 80%, 90%, 95% reduction or complete inhibition of the regulation of the allosteric phosphofructokinase by fructose-2,6-bisphosphate or fructose-1,6-bisphosphate.

As used herein, “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, “blood sample” includes whole blood, serum, and plasma.

As used herein, “serum” refers to the fluid portion of the blood obtained after removal of the fibrin clot and blood cells, distinguished from the plasma in circulating blood.

As used herein, “plasma” refers to the fluid, noncellular portion of the blood, distinguished from the serum obtained after coagulation.

As used herein, “fluid” refers to any composition that can flow. Fluids thus encompass compositions that are in the form of semi-solids, pastes, solutions, aqueous mixtures, gels, lotions, creams and other such compositions.

As used herein, “contacting” means bringing two or more components together. “Contacting” can be achieved by mixing all the components in a fluid or semi-fluid mixture. “Contacting” can also be achieved when one or more components are brought into contact with one or more other components on a solid surface such as a solid tissue section or a substrate.

As used herein, “antibody” is used in the broadest sense. Therefore, an “antibody” can be naturally occurring or man-made such as monoclonal antibodies produced by conventional hybridoma technology and/or a functional fragment thereof. Antibodies of the present invention comprise monoclonal and polyclonal antibodies as well as fragments (such as Fab, Fab′, F(ab′)₂, Fv) containing the antigen-binding domain and/or one or more complementary determining regions of these antibodies.

As used herein, “monoclonal antibody” refers to an antibody obtained from a population of substantially homogeneous antibodies, i.e., the antibodies comprising the population are identical except for possible naturally occurring mutations that are present in minor amounts. As used herein, a “monoclonal antibody” further refers to functional fragments of monoclonal antibodies.

As used herein, “small molecule” refers to a molecule that, without forming homo-aggregates or without attaching to a macromolecule or adjuvant, is incapable of generating an antibody that specifically binds to the small molecule. Preferably, the small molecule has a molecular weight that is about or less than 10,000 daltons. More preferably, the small molecule has a molecular weight that is about or less than 5,000 dalton.

As used herein the term “assessing” is intended to include quantitative and qualitative determination in the sense of obtaining an absolute value for the amount or concentration of the analyte present in the sample, and also of obtaining an index, ratio, percentage, visual or other value indicative of the level of analyte in the sample. Assessment may be direct or indirect and the chemical species actually detected need not of course be the analyte itself but may for example be a derivative thereof or some further substance.

As used herein, “production by recombinant means” refers to production methods that use recombinant nucleic acid methods that rely on well known methods of molecular biology for expressing proteins encoded by cloned nucleic acids. See e.g., Current Protocols in Molecular Biology (Ausubel et al. eds., Wiley Interscience Publishers, 1995); Molecular Cloning: A Laboratory Manual (J. Sambrook, E. Fritsch, T. Maniatis eds., Cold Spring Harbor Laboratory Press, 2d ed. 1989).

As used herein, “vector (or plasmid)” refers to discrete elements that are used to introduce heterologous DNA into cells for either expression or replication thereof. Selection and use of such vehicles are well known within the skill of the artisan. An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes include the bacteriophage T7 and T3 promoters, and the like.

As used herein, “operatively linked or operationally associated” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus ribosome binding sites (see, e.g., Kozak, J. Biol. Chem., 266:19867-19870 (1991)) can be inserted immediately 5′ of the start codon and may enhance expression. The desirability of (or need for) such modification may be empirically determined.

As used herein, “complementary” when referring to two nucleic acid molecules, means that the two sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein: “stringency of hybridization” in determining percentage mismatch is as follows:

1) high stringency: 0.1×SSPE, 0.1% SDS, 65° C.;

2) medium stringency: 0.2×SSPE, 0.1% SDS, 50° C. (also referred to as moderate stringency); and

3) low stringency: 1.0×SSPE, 0.1% SDS, 50° C.

It is understood that equivalent stringencies may be achieved using alternative buffers, salts and temperatures.

The term “substantially” identical or homologous or similar varies with the context as understood by those skilled in the relevant art and generally means at least 70%, preferably means at least 80%, more preferably at least 90%, and most preferably at least 95% identity.

As used herein, high-throughput screening (HTS) refers to processes that test a large number of samples, such as samples of diverse chemical structures against disease targets to identify “hits” (see, e.g., Broach, et al., High throughput screening for drug discovery, Nature, 384:14-16 (1996); Janzen, et al., High throughput screening as a discovery tool in the pharmaceutical industry, Lab Robotics Automation: 8261-265 (1996); Fernandes, P. B., Letter from the society president, J. Biomol. Screening, 2:1 (1997); Burbaum, et al., New technologies for high-throughput screening, Curr. Opin. Chem. Biol., 1:72-78 (1997)). HTS operations are highly automated and computerized to handle sample preparation, assay procedures and the subsequent processing of large volumes of data.

Methods and Kits for Assaying an Analyte in a Sample

In one aspect, a method for assaying an analyte in a sample is provided, which method comprises: a) contacting a sample containing an analyte or suspected of containing an analyte with a specific binding reagent for said analyte in the presence of an allosteric phosphofructokinase and an analyte or analyte analog conjugated to fructose-2,6-bisphosphate or fructose-1,6-bisphosphate or a compound comprising fructose-2,6-bisphosphate or fructose-1,6-bisphosphate, under conditions such that binding of said specific binding reagent to said analyte or analyte analog conjugated to said fructose-2,6-bisphosphate or fructose-1,6-bisphosphate or said compound comprising fructose-2,6-bisphosphate or fructose-1,6-bisphosphate prevents or reduces regulation of said allosteric phosphofructokinase by said fructose-2,6-bisphosphate or fructose-1,6-bisphosphate or said compound comprising fructose-2,6-bisphosphate or fructose-1,6-bisphosphate, and said analyte, if present in said sample, competes with said analyte or analyte analog conjugated to said fructose-2,6-bisphosphate or fructose-1,6-bisphosphate or said compound comprising fructose-2,6-bisphosphate or fructose-1,6-bisphosphate, for binding with said specific binding reagent and reduces or prevents binding of said specific binding reagent to said analyte or analyte analog conjugated to said fructose-2,6-bisphosphate or fructose-1,6-bisphosphate or said compound comprising fructose-2,6-bisphosphate or fructose-1,6-bisphosphate, leading to increased regulation of said allosteric phosphofructokinase; and b) determining the presence, absence and/or amount of said analyte in said sample by assessing activity of said allosteric phosphofructokinase.

In another aspect, a kit for assaying an analyte in a sample is provided, which kit comprises: a) a specific binding reagent for an analyte; b) an allosteric phosphofructokinase; c) an analyte or analyte analog conjugated to fructose-2,6-bisphosphate or fructose-1,6-bisphosphate or a compound comprising fructose-2,6-bisphosphate or fructose-1,6-bisphosphate; and d) means for assessing activity of said allosteric phosphofructokinase.

The present methods and kits can be used to assay any suitable sample. In one example, the present methods and kits can be used to assay a biosample, e.g., a blood or urine sample.

The present methods and kits can be used to assay any suitable analyte. In one example, the present methods and kits can be used to assay a small molecule, a protein, a peptide, a hormone, a nucleic acid, a oligonucleotide, a fatty acid, a saccharide or a polysaccharide. In another example, the present methods and kits can be used to assay thyroid hormones such as T3 and T4.

Any suitable specific binding reagent can be used in the present methods and kits. For example, a small molecule, a protein, a peptide, a hormone, a nucleic acid, a oligonucleotide, a fatty acid, a saccharide or a polysaccharide can be used as a specific binding reagent in the present methods and kits. In another example, an antibody or a soluble receptor is used as the specific binding reagent.

Any suitable allosteric enzyme can be used in the present methods and kits. For example, a wild-type allosteric enzyme or a mutant allosteric enzyme derived from an allosteric enzyme can be used, or a mutant allosteric enzyme derived from a non-allosteric enzyme can be used. The allosteric enzyme can be a prokaryotic enzyme or an eukaryotic enzyme. The allosteric enzymes can be prepared by any suitable methods, e.g., recombinant methods, chemical synthesis or a combination thereof.

An allosteric enzyme is an enzyme whose catalytic function may be modified by interaction with small molecules, not only at its active site, but also at a spatially distinct (allosteric) site of different specificity. An allosteric regulator (or effector) is a molecule bound at such a site that increases or decreases the activity of the enzyme.

In one specific example, the allosteric enzyme is an allosteric phosphofructokinase. An allosteric phosphofructokinase refers to an enzyme, or a functional fragment or derivative thereof, that converts fructose 6-phosphate to fructose 1,6-bisphosphate. The phosphofructokinase can be ATP or pyrophosphate dependent. Any suitable allosteric phosphofructokinase can be used. The allosteric phosphofructokinase can be a native or a recombinant phosphofructokinase.

For example, the allosteric phosphofructokinase can be a wild-type allosteric phosphofructokinase or a mutant allosteric phosphofructokinase derived from an allosteric phosphofructokinase, or a mutant allosteric phosphofructokinase derived from a non-allosteric phosphofructokinase. In another example, the allosteric phosphofructokinase can be a phosphofructokinase from D. discoideum (Dd), mouse liver (Ml), rabbit muscle (Rm), ascites tumor cells (C-type) (At), S. cerevisiae (Sc), Bacillus stearothermophilus (Bs), or Escherichia coli (Ec). In still another example, the allosteric phosphofructokinase can be a phosphofructokinase disclosed in the following patents, patent applications and non-patent publications: U.S. Pat. Nos. 6,806,068, 6,737,237, 6,699,654, 6,596,851, 6,413,939, 6,255,046, 5,583,011, 5,387,756, 5,356,787, 5,218,091, 4,959,212, 4,897,353, 4,812,398, 4,806,343, 4,774,088, 4,710,460, 4,599,311; U.S. patent application Nos. US2003/228568, US2003/186352, and US2003/092137; PCT applications WO 98/03661 and WO 97/05158; Santamaria et al., J. Biol. Chem., 277(2):1210-6 (2002); Kemp and Foe, Mol. Cell. Biochem., 57(2):147-54 (1983); Martinez-Costa et al., Biochem J, 377(Pt 1):77-84 (2004); and Strausberg et al., Proc. Natl. Acad. Sci. U.S.A., 99(26):16899-16903 (2002) (GenBank Accession Nos. BC00779 and BC004920).

In yet another example, the allosteric phosphofructokinase can be an allosteric phosphofructokinas from D. discoideum. The D. discoideum allosteric phosphofructokinase can have a C-terminus deletion of 1 to 36 amino acid residues, i.e., the deletions of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 residues from the C-terminus. The D. discoideum allosteric phosphofructokinase can also have a point mutation at the very C-terminus amino acid residue, e.g., a mutation from Leu to any of the other natural 19 amino acid residues, such as a Leu to Ala change.

Any suitable allosteric regulator that is compatible with the allosteric enzyme can be used in the present methods and kits. For example, if the allosteric enzyme is an allosteric phosphofructokinase, fructose-2,6-bisphosphate or fructose-1,6-bisphosphate or a compound comprising fructose-2,6-bisphosphate or fructose-1,6-bisphosphate can be used as the allosteric regulator. In another example, a 1-substituted-β-D-fructofuranose 2,6-bisphosphate compound is used as the allosteric regulator, which fructose-2,6-bisphosphate compound has the following formula I:

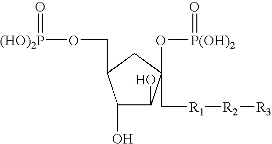

wherein R₁ is O, N or S; R₂ is a C₃ to C₃₀ alkyl group; and R₃ is OH, SH, NH₂, COOH, CONH₂, or COOR₄, wherein R₄ is a C₁ to C₃₀ alkyl group.

The analyte or analyte analog can be conjugated to the allosteric regulator by any suitable methods. For example, the analyte or analyte analog can be conjugated to the allosteric regulator covalently or non-covalently, e.g., by absorption. In a specific example, the analyte or analyte analog can be conjugated to the allosteric regulator via a linker. Any suitable linker can be used, e.g., a 2-40 carbon and/or heteroatom chain.

The phosphofructokinase activity can be determined by any suitable methods. For example, phosphofructokinase activity can be determined by any suitable methods disclosed in U.S. Pat. Nos. 6,806,068, 6,737,237, 6,699,654, 6,596,851, 6,413,939, 6,255,046, 5,583,011, 5,387,756, 5,356,787, 5,218,091, 4,959,212, 4,897,353, 4,812,398, 4,806,343, 4,774,088, 4,710,460, 4,599,311; U.S. patent application Nos. US2003/228568, US2003/186352, and US2003/092137; PCT applications WO 98/03661 and WO 97/05158; Santamaria et al., J. Biol. Chem., 277(2):1210-6 (2002); Kemp and Foe, Mol. Cell. Biochem., 57(2):147-54 (1983); Martinez-Costa et al., Biochem J., 377(Pt 1):77-84 (2004); and Strausberg et al., Proc. Natl. Acad. Sci. U.S.A., 99(26):16899-16903 (2002).

In another example, the phosphofructokinase activity is determined by measuring enzymatic conversion between NAD and NADH, or enzymatic conversion between NADP and NADPH.

In one specific embodiment, the following reaction scheme is used to measure phosphofructokinase activity: 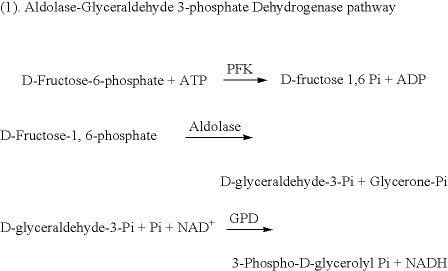
Where PFK is 6-phosphofructokinase, Aldolase is fructose-bisphosphate aldolase and GPD is glyceraldehyde-3-phosphate dehydrogenase.

In this embodiment, the activity of the phosphofructokinase can be assessed by measuring NAD⁺ consumed or NADH generated in the presence of D-fructose-6-phosphate, ATP, phosphate, and NAD⁺.

The phosphofructokinase activity can also be assessed by measuring NAD⁺ consumed or NADH generated in the reactions catalyzed by fructose-bisphosphate aldolase and glyceraldehyde-3-phosphate dehydrogenase.

Any suitable fructose-bisphosphate aldolase can be used. For example, fructose-bisphosphate aldolase disclosed in the following patents and patent applications can be used: U.S. Pat. Nos. 5,162,221, 6,555,353, 6,767,744, 6,773,905 and 6,908,992, U.S. patent application Nos. US2004/073976 and US2003/087283, and PCT application WO 02/48188.

Any suitable glyceraldehyde-3-phosphate dehydrogenase can be used. For example, glyceraldehyde-3-phosphate dehydrogenase disclosed in the following patents and patent applications can be used: U.S. Pat. Nos. 5,349,059, RE35,749, 6,348,331, 6,410,230, 6,440,720, 6,521,743 and 6,613,547, and U.S. patent application No. 2005/0266474.

In another specific embodiment, the following reaction scheme is used to measure phosphofructokinase activity: 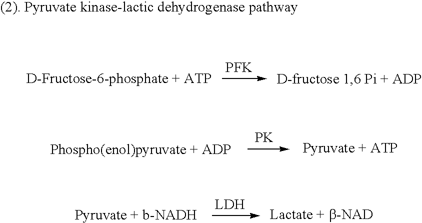
Where PFK is 6-phosphofructokinase, PK is pyruvate kinase, LDH is lactate dehydrogenase.

In this embodiment, the activity of the phosphofructokinase can be assessed by measuring NADH consumed or NAD⁺ generated in the presence of D-fructose-6-phosphate, ATP, phospho(enol)pyruvate and b-NADH.

The activity of the phosphofructokinase can also be assessed by measuring NADH consumed or NAD⁺ generated in the reactions catalyzed by pyruvate kinase and lactate dehydrogenase.

Any suitable pyruvate kinase can be used. For example, pyruvate kinase disclosed in the following patents and patent applications can be used: U.S. Pat. Nos. 4,331,762, 4,349,631, 6,214,879 and 6,534,501, U.S. patent application No. 2004/0152648, and PCT application WO 90/05917.

Any suitable lactate dehydrogenase can be used. For example, lactate dehydrogenase disclosed in the following patents and patent applications can be used: U.S. Pat. Nos. 4,258,131, 4,324,860, 6,057,141, 6,268,189, 6,503,743 and 6,753,174, U.S. patent application No. 2002/055152 A1, and PCT applications WO 03/076616, WO 98/01545 and WO 94/24287.

In yet another specific embodiment, the following reaction scheme is used to measure phosphofructokinase activity: 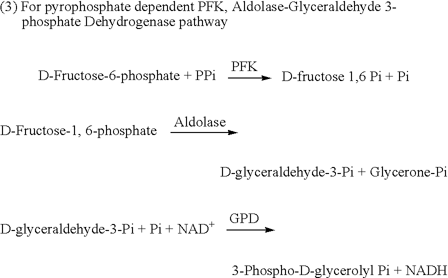
Where PFK is 6-phosphofructokinase, Aldolase is fructose-bisphosphate aldolase and GPD is glyceraldehyde-3-phosphate dehydrogenase.

In this embodiment, the activity of the phosphofructokinase can be assessed by measuring NAD⁺ consumed or NADH generated in the presence of D-fructose-6-phosphate, pyrophosphate (ppi), phosphate, and NAD⁺. The phosphofructokinase can also be assessed by measuring NAD⁺ consumed or NADH generated in the reactions catalyzed by fructose-bisphosphate aldolase and glyceraldehyde-3-phosphate dehydrogenase.

In another embodiment, the activity of the phosphofructokinase can be assessed by measuring NADH consumed or NAD generated in the presence of D-fructose-6-phosphate, pyrophosphate, aldolase, triose phosphate isomerase and glycerol 3-phosphate dehydrogenase.

In yet another specific embodiment, the following reaction scheme is used to measure phosphofructokinase activity: 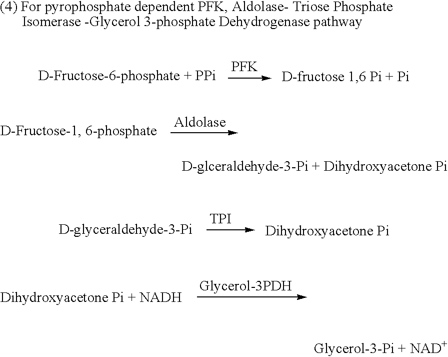
Where PFK is 6-phosphofructokinase, Aldolase is fructose-bisphosphate aldolase, TPI is triose phosphate isomerase, and Glycerol-3PDH is glycerol-3-phosphate dehydrogenase.

In this embodiment, the activity of the phosphofructokinase can be assessed by measuring NADH consumed or NAD⁺ generated in the presence of D-fructose-6-phosphate, pyrophosphate (ppi), triose phosphate isomerase and glycerol-3-phosphate dehydrogenase.

Any suitable triose phosphate isomerase can be used. For example, triose phosphate isomerase in the following patents and patent applications can be used: U.S. Pat. Nos. 5,218,091, 5,024,941, 4,599,311, 4,171,244 and 4,043,872, U.S. patent application No. 2004/0038351.

Any suitable glycerol-3-phosphate dehydrogenase can be used. For example, glycerol-3-phosphate dehydrogenase in the following patents and patent applications can be used: U.S. Pat. No. 6,103,520, U.S. patent application Nos. 20050239179 and 20020034794.

Any suitable means for assessing activity of the phosphofructokinase activity can be used in the present methods and kits. For example, such means can comprise enzyme(s) or substrate(s) for effecting and measuring conversion between NAD and NADH, or enzymatic conversion between NADP and NADPH. In one example, the means for assessing activity of the allosteric phosphofructokinase comprises D-fructose-6-phosphate, ATP, phosphate, and NAD⁺. In another example, the means for assessing activity of the allosteric phosphofructokinase comprises fructose-bisphosphate aldolase and glyceraldehyde-3-phosphate dehydrogenase. In still another example, the means for assessing activity of the allosteric phosphofructokinase comprises D-fructose-6-phosphate, ATP, phospho(enol)pyruvate and b-NADH. In yet another example, the means for assessing activity of the allosteric phosphofructokinase comprises pyruvate kinase and lactic acid (or lactate) dehydrogenase. In yet another example, the means for assessing activity of the allosteric phosphofructokinase comprises D-fructose-6-phosphate, pyrophosphate (ppi), phosphate, and NAD⁺.

The present methods and kits can be used in any suitable assay format. For example, the present methods and kits can be used in a homogeneous assay. Alternatively, the present methods and kits can be used in a heterogeneous assay, in which one or more of the a specific binding reagent for an analyte, an allosteric enzyme, and an analyte or analyte analog conjugated to an allosteric regulator of the enzyme can be bound or attached on a suitable solid support, e.g., a microtiter plate, a bead, a tube, a chip, etc.

The present methods and kits can be used for assaying a single analyte. Alternatively, the present methods and kits can be used for assaying multiple analytes, whether sequentially or simultaneously. For example, the present methods and kits can be used for assaying multiple analytes, whether sequentially or simultaneously in a high throughput format. The present methods and kits can be used manually or used in an automated format.

The present methods and kits can be used for any suitable purpose. For example, the present methods and kits can be used for clinical prognosis, diagnosis and/or treatment monitoring. In another example, the present methods and kits can be used for drug screening.

In yet another aspect, the present invention is directed to an isolated nucleic acid fragment, which isolated nucleic acid fragment comprises a sequence of nucleotides encoding a mutant D. discoideum allosteric phosphofructokinase having a C-terminus deletion in the 1 to 36 amino acid residues, i.e., the deletions of 3, 5, 6, 7, 9, 10, 11, 12, 13, 15, 16, 17, 18, 19, 20, 21, 22, 23, 24, 25, 27, 28, 29, 30, 31, 32, 33, 34, or 35 residues from the C-terminus; or a mutant D. discoideum allosteric phosphofructokinase having a point mutation at the very C-terminus amino acid residue, e.g., a mutation from Leu to any of the other natural 18 amino acid residues, except a Leu to Ala change.

The isolated nucleic acid fragment can be in any suitable forms, e.g., DNA, RNA, PNA, etc., or a combination thereof. A plasmid comprising the above isolated nucleic acid fragment is also provided. A cell comprising the above plasmid is also provided. Any suitable cells can be used. For example, the cell can be a bacterial cell, a yeast cell, a fungal cell, a plant cell, an insect cell or an animal cell. A method for producing a mutant D. discoideum allosteric phosphofructokinase is also provided, which method comprises growing the above cell under conditions whereby the mutant D. discoideum allosteric phosphofructokinase is expressed by the cell, and recovering the expressed mutant D. discoideum allosteric phosphofructokinase.

In yet another aspect, the present invention is directed to a substantially purified D. discoideum allosteric phosphofructokinase, having a C-terminus deletion in the 1 to 36 amino acid residues, i.e., the deletions of 3, 5, 6, 7, 9, 10, 11, 12, 13, 15, 16, 17, 18, 19, 20, 21, 22, 23, 24, 25, 27, 28, 29, 30, 31, 32, 33, 34, or 35 residues from the C-terminus; or a mutant D. discoideum allosteric phosphofructokinase having a point mutation at the very C-terminus amino acid residue, e.g., a mutation from Leu to any of the other natural 18 amino acid residues, except a Leu to Ala change.

The isolated nucleic acid encoding a mutant D. discoideum allosteric phosphofructokinase or the substantially purified D. discoideum allosteric phosphofructokinase can be prepared by any suitable methods, e.g., recombinant methods, chemical synthesis or a combination thereof.

1-Substituted-β-D-Fructofuranose 2,6-Bisphosphate Compound

In another aspect, a 1-substituted-β-D-fructofuranose 2,6-bisphosphate compound, or a salt thereof, is provided, which compound, or a salt thereof, has the following formula I:

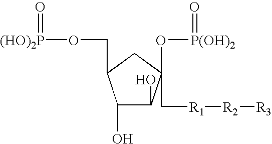

wherein R₁ is O, N or S; R₂ is a C₃ to C₃₀ alkyl group; and R₃ is OH, SH, NH₂, COOH, CONH₂, or COOR₄, wherein R₄ is a C₁ to C₃₀ alkyl group.

In one example, R₁ is 0 and R₃ is OH, SH, NH₂, COOH, CONH₂, or COOR₄. In another example, R₁ is N and R₃ is OH, SH, NH₂, COOH, CONH₂, or COOR₄. In still another example, R₁ is S and R₃ is OH, SH, NH₂, COOH, CONH₂, or COOR₄.

The C₃ to C₃₀ alkyl group can be any suitable aliphatic group including alkane, alkene, alkyne and cyclic aliphatic groups.

The compounds of the present invention can be a particular stereoisomer, e.g., R- or S-configuration, or a mixture thereof, e.g., a racemic mixture. The term compounds contemplated herein encompasses all diagnostically or pharmaceutically active species of the compounds, or solutions thereof, or mixtures thereof. The compounds contemplated herein also encompass hydrated versions, such as aqueous solutions, hydrolyzed products or ionized products of these compounds; and these compounds may contain different number of attached water molecules.

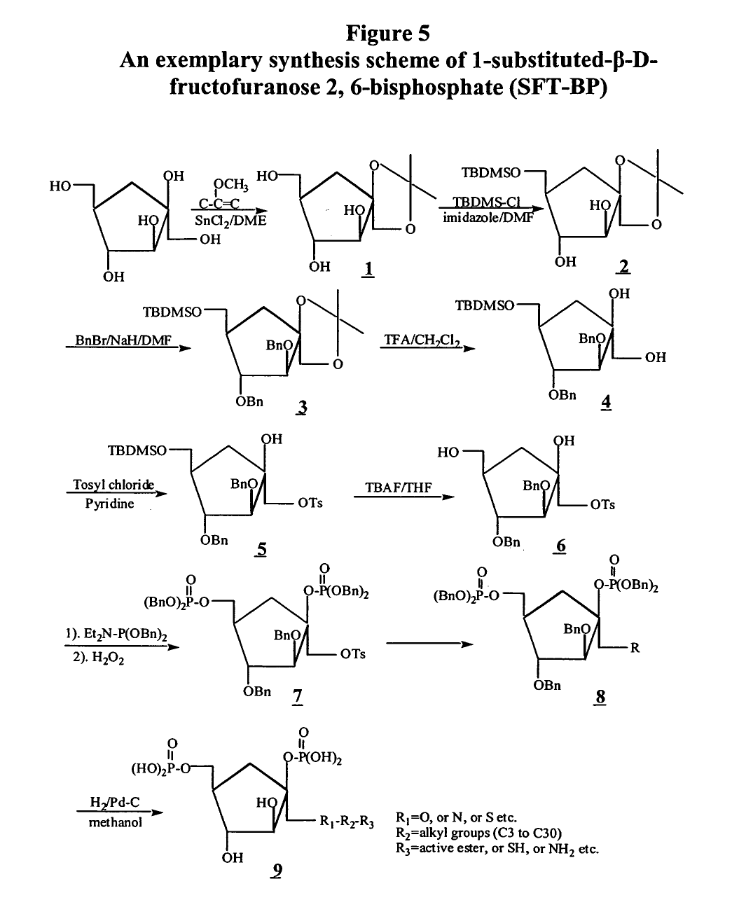

The compounds of the present invention can be prepared or synthesized according to any suitable methods. Preferably, synthetic methods illustrated in the following Example Section and FIG. 5 are used to prepare the compounds.

Also preferably, the compound, or its diagnostically or pharmaceutically acceptable salt thereof, is provided in the form of a diagnostic or pharmaceutical composition, either alone or in combination with a diagnostically or pharmaceutically acceptable carrier or excipient.

The compounds of the present invention can be prepared as their diagnostically or pharmaceutically acceptable salts with any suitable acids. For example, inorganic acids, such as hydrochloric acid, hydrobromic acid, nitric acid, sulfuric acid, and phosphoric acid, etc., can be used. In another example, organic acids, such as formic acid, acetic acid, propanoic acid, benzoic acid, maleic acid, fumaric acid, succinic acid, tartaric acid, citric acid, etc., can be used. In still another example, alkyl sulfonic acid, such as methyl sulfonic acid, ethyl sulfonic acid, etc., can be used. In yet another example, aryl sulfonic acid, such as benzene sulfonic acid, p-toluene sulfonic acid, etc, can be used.

An analyte, analyte analog or a specific binding partner for an analyte conjugated to the 1-substituted-β-D-fructofuranose 2,6-bisphosphate compound is also provided.

Any suitable analyte or analyte analog can be used in the present conjugates. In one example, the analyte or analyte analog can be a small molecule, a protein, a peptide, a hormone, a nucleic acid, a oligonucleotide, a fatty acid, a saccharide or a polysaccharide. In another example, the analyte is thyroid hormone T4.

Any suitable specific binding reagent can be used in the present conjugates. For example, a small molecule, a protein, a peptide, a hormone, a nucleic acid, a oligonucleotide, a fatty acid, a saccharide or a polysaccharide can be used as a specific binding reagent in the present conjugates. In another example, an antibody or a soluble receptor is used as the specific binding reagent in the present conjugates.

Kits comprising the conjugate, and methods using the conjugate for assaying an analyte are further provided. Preferably, the conjugates are used in methods for assaying an analyte as described in the previous section.

### EXEMPLARY EMBODIMENTS

Phosphofructokinase (EC 2.7.1.11) or its genetically engineered mutants are allosteric enzymes. These enzymes are less active in the absence of certain allosteric regulators, and are highly activated when the allosteric regulators are present. The allosteric regulators for phosphofructokinase or its mutants can be fructose bisphosphate such as fructose-2,6-bisphosphate and fructose-1,6-bisphosphate, or ATP or AMP. In this example, analogs of the allosteric regulators are made by attaching a linker (2-40 carbon chain) to fructose-2,6-bisphosphate or fructose-1,-6-bisphosphate in such a way that the linker attached regulators are still functionally active. Meanwhile, the other end of the linker contains a chemically reactive group that can form a covalent conjugate with an analyte molecule including small molecules, proteins, peptides, hormones, nucleic acids, fatty acids, and saccharides. The analyte conjugated regulator (briefly conjugated analyte) is then used as a competitor of the free analyte in samples for binding with an antibody specific for the analyte.

This example provides an allosteric enzyme coupled immunoassay (AECIA) technology that provides a universal assay format for various analytes with a uniformed detection method. In the assay format, a competing analyte, such as Thyroid hormone T4, is chemically conjugated to an allosteric regulator, such as fructose-2,6-bisphosphate. The conjugated analyte binds to the allosteric side of the allosteric enzyme, phosphofructokinase, which results in activation of the enzyme and therefore enhanced signal generation. Binding of an antibody to the conjugated analyte sterically hinders its binding to the allosteric side of the enzyme, and therefore no signal or less signal is generated.

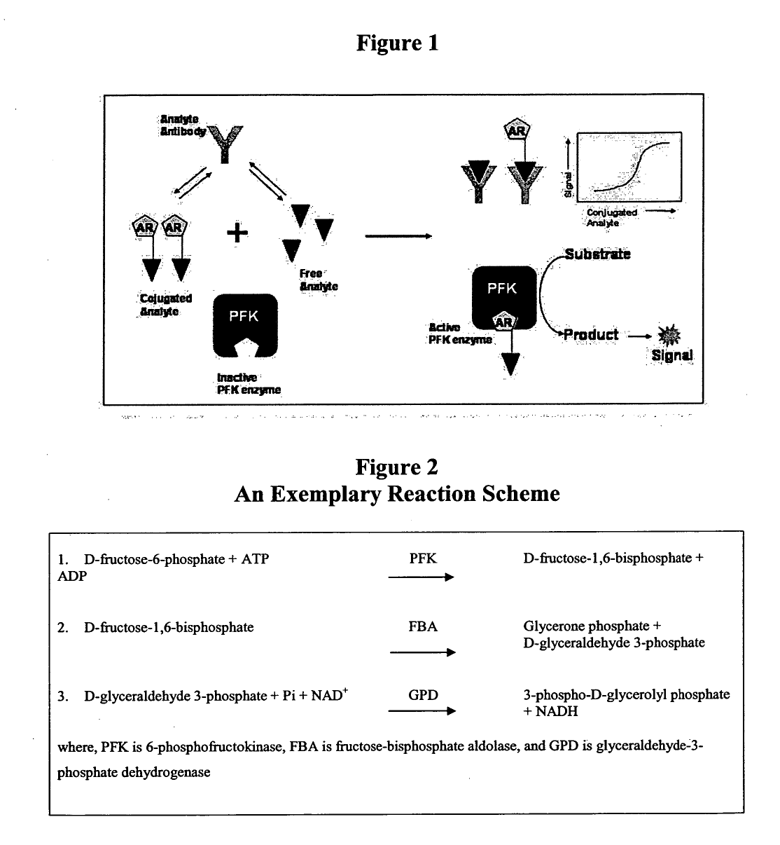

Competitive displacement of the conjugated analyte from the antibody occurs in the presence of free analyte, allowing it to freely bind to the allosteric side of phosphofructokinase and generate signal. The principle of an exemplary AECIA is depicted in the FIG. 1.

The exemplary AECIA technology can be widely used for detection of analytes that are markers of clinical diagnostics or targets of drug discovery. Since the AECIA can be used in a homogenous assay format, it can be easily applied to various automated clinical chemistry analyzers. Since the AECIA format is scalable to 96,384, and 1536-well formats, it can also be used for high throughput screening in search for drug candidates.

An important feature of the exemplary AECIA assay is that signal, generated by phosphofructokinase turnover, is directly proportional to the concentration of the analyte in a sample. The greater the signal generation is, the higher the analyte concentration will be if a fixed amount of the analyte specific antibody is present. Another feature of the AECIA assay is that the signal detection method is universal for all analytes, which significantly simplifies instrumentational requirements and parameter settings. The signal detection of phosphofructokinase activity can be based on the formation of the co-enzyme NADH, and the exemplary enzyme reaction scheme is indicated in FIG. 2.

### EXAMPLES

Materials. All chemicals, reagents, and enzymes used were obtained from Roche Molecular Diagnostics, Invitrogen, Amersham Bioscience and Sigma.

### Example 1

### Activation of Potato Tuber Pyrophosphate-Dependent PFK by Fructose-2,6-Bisphosphate

Purification of Pyrophosphate-Dependent Fructose-6-Phosphate Kinase (Ec2.7.1.90) (PPi-PFK) from Potato Tubers

Potato tubers PPi-PFK was purified according to the procedure described in Van Schafingen et al., Eur. J. Biochem., 129:191-195 (1982). Peeled potato tubers (330 g) were homogenized in a blendor with 2 volume of ice-cold 20 mM Hepes pH8.2 containing 20 mM potassium acetate and 2 mM dithiothreitol. The homogenate was filtered through cheese cloth. Solid sodium pyrophosphate and 1 mM MgCl2 were added to the filtrate to reach 2 mM and the pH was adjusted to 8.2 at 0° C. The mixture was brought to 59° C. by shaking in a water bath adjusted to 70° C. and maintained for 5 min in a water bath at 59° C.; it was then cooled and brought to pH 7.1 at 0° C. All subsequent steps were carried out at 0-4° C. Six (6) g of poly(ethylene glycol) 6000 were added per 100 ml of solution and after mixing for 15 min, the preparation was allowed to stand for 10 min and then centrifuged for 10 min at 4000×g; 8 g of poly(ethylene glycol) were then added per 100 ml of the resulting supernatant (700 ml) which was mixed and centrifuged as described above. The resulting pellet was dissolved in 40 ml of a solution containing 20 mM Tris, pH8.2, 20 mM KCl, and 2 mM dithiothreitol and applied to a column (25×120 mm) of DEAE-cellulose equilibrated with the same buffer. The column was washed with 100 ml of buffer and developed with a gradient of KCl (20-400 mM) in the same dithiothreitol/Tris buffer. The enzyme was eluted as a single peak at a concentration of 120 mM KCl. The most effective fractions were pooled, mixed with 1 volume of cold glycerol and kept at −20° C.

Stimulation Assay of Potato Tuber PPi-PFK by Fructose 2,6 Pi

The stimulation assay of potato tuber PPi-PFK by Fructose 2,6 Pi was conducted according to the following procedure:

(1). Make F2, 6Pi Stock solution

(1). Make 10 mM F2,6Pi 1 mL

To 1 mL 50 mM MES PH6.5 containing 30% glycerol, add

0.01M×1 mL×428/0.95=4.5 mg of 95% D-Fructose 2,6Pi (Sigma F7006)

(2). Dilute 10 mM F2,6Pi stock solution to 100 μM, 100 μM and 1 μM

A. 100 mM F26Pi:

10 μL 10 mM F26Pi+990 μL 50 mM MES PH6.5 containing 30% glycerol

B. 10 μM F26Pi:

100 μL 100 μM F26Pi (A)+900 μL 50 mM MES PH6.5 containing 30% glycerol

C. 1 μM F26Pi:

100 μL 10 μM (B)+900 μL 50 mM MES PH6.5 containing 30% glycerol

(2). Assay Buffer Mixture 10 mL

TABLE 1
Assay buffer mixture for stimulation assay of
potato tuber PPi-PFK (10 mL)

| Component Concentration | Stock Solution | Amount |
|---|---|---|
| 50 mM TrisCl pH8.0 | 1M TrisCl pH8.0 stock | 0.5 mL |
| 5 mM MgCl₂ | 1M MgCl₂ stock | 50 μL |
| 0.15 mM NADH | 100 mM NADH stock | 15 μL |
| 100 μg/mL Aldolase | Powder | 1 mg(weigh out) |
| 20 μg/mL GPD/TPI | 10 mg/mL cocktail stock | 20 μL |
| cocktail | | |
| 1 mM F6Pi | 100 mM F6Pi stock | 0.1 mL |
| 0.2% Casein | 10% stcok | 0.2 mL |
| 0.1% γ-HPCD | 10% stock | 0.1 mL |
| 0.05% Brij 35 | 30% stock | 17 μL |

(3). To 2 mL of assay buffer mixture above, add 40 μL of potato PFK stock in 30% glycerol (3 units/mL), and mix well.
(4). Start the assay by adding different concentrations of F2,6Pi: F2,6Pi at indicated concentration and lastly 2.5 mM PPi.

150 μL reaction per assay. Record OD340 nm for 300 seconds.

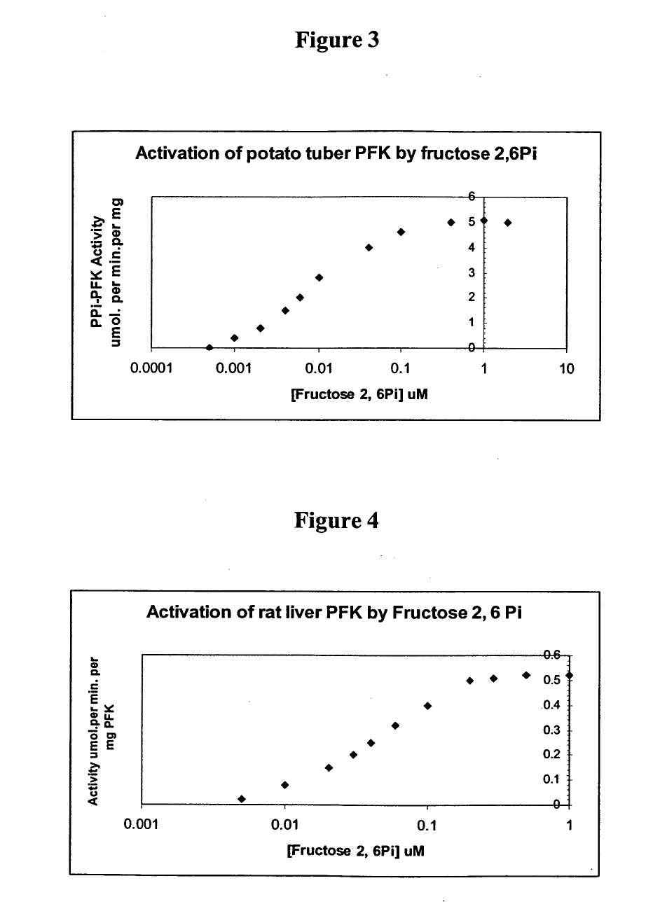

As shown in FIG. 3, in the presence of 1 mM fructose 6-phosphate and 2.5 mM PPi, a half-maximal effect was obtained at approximately 5.5 nM of fructose 2, 6 Pi.

### Example 2

### Activation of ATP-Dependent Rat Liver PFK by Fructose-2,6-Bisphosphate

Purification of ATP-Dependent Fructose-6-Phosphate Kinase (EC2.7.1.11) from Rat Livers

ATP-dependent fructose-6-phosphate kinase (EC2.7.1.11) was purified from rat livers according to the procedures described in Reinhart and Lardy., Biochemistry, 19:1477-1484 (1980). The purification procedure includes a heat step, ammonium sulfate precipitation, and DEAE ion-exchange and gel filtration chromatography as follows:

Homogenization. Fresh livers from rats were homogenized in a homogenizer with 3 volumes of chilled buffer containing 50 mM Tris-HCl, 50 mM NaF, 5 mM DTT, and 1 m mM ATP, pH 8.0. The homogenate was then spun at 100,000 g for 60 min and the resulting supernatant solution was poured through glass wool.

Heat Treatment. The solution was brought to 60° C. in a boiling-water bath with continuous stirring. The suspension was kept at 60° C. for 3 min and then cooled to less than 10° C. by swirling in an ice-salt bath. After centrifugation for 30 min at 20,000 g, the supernatant solution was once again decanted through glass wool.

Ammonium Sulfate Precipitation. A total of 18.5 g of enzyme-grade ammonium sulfate per 100 mL of solution was added slowly. Once the salt was completely dissolved, the mixture was kept without stirring at 4° C. for 90 min and then centrifuged for 20 min at 17,000 g, and the supernatant solution was discarded. The pellet was resuspended in a minimum volume of column buffer (20 mM KPi, 3 mM MgSO₄, 5 mM DTT, 20 μM EDTA, and 1 mM F6P, pH7.6). The suspension, which was not entirely soluble, was dialyzed overnight against this buffer and centrifuged at 2000 g for 15 min, and the pellet was discarded.

DEAE chromatography. DEAE column was equilibrated with the column buffer (20 mM KPi, 3 mM MgSO₄, 5 mM DTT, 20 μM EDTA, and 1 mM F6P, pH7.6). The enzyme sample from step (3) diluted to a protein concentration of less than 5 mg/mL, was applied to the column. The column was then washed with the column buffer until no more protein was eluted. The PFK was eluted from the column with a linear gradient made from equal volumes of the column buffer and buffer containing 300 mM KPi, 3 mM MgSO₄, 5 mM DTT, 20 μM EDTA and 1 mM F6Pi, pH7.6. The fractions containing significant PFK activity were pooled and concentrated by precipitating the protein with ammonium sulfate (final concentration was 55% of saturation). The pellet obtained by centrifuge at 17,000 g for 30 min was resuspended in 3-4 mL of column buffer. This suspension was spun at 2000 g for 15 min to clarify.

Sepharose 2B Chromatography. The solution was layered and was loaded onto a Sepharose 2B column measuring 36×4 cm and eluted with column buffer. Three (3) mL fractions were collected. The PFK activity eluted soon after the void volume. For maximum purity, a conservative cut was made by pooling only the fractions within the Gaussian portion of the peak. These pooled factions were quickly concentrated to a volume of 8 mL with the centricon. The enzyme was then dialyzed overnight against storage buffer containing 20 mM KPi, 3 mM MgSO4, 1 mM fructose-6-Phosphate (F6Pi), 5 mM DTT, 100 mM EDTA, and 20% glycerol, pH7.6.

Stimulation Assay of Rat Liver Phosphofructokinase by Fructose 2,6 Pi

The assay was performed at pH 7.1 and 22° C. The incubation mixture contained 100 mM KCl, 1 mM NH₄Cl, 0.15 mM NADH, 50 mM Hepes buffer, 50 μg/ml aldolase, 10 μg/ml glycerol-3-phosphate dehydrogenase, 1 μg/ml triose phosphate isomerase, 5 mM MgCl2, 1 mM AMP, 2 mM fructose 6-phosphate, and 0.2 mM Pi. The reaction was initiated by adding 3 mM ATP. As shown in FIG. 4, in the presence of 2 mM fructose 6-phosphate and 1 mM AMP, a half-maximal effect was obtained at approximately 0.04 μM fructose 2, 6 Pi.

### Example 3

### Genetically Creating an Allosteric Phophofructokinase Use Thereof

An allosteric phophofructokinase was genetically created from a nonallosteric enzyme from Dictyostelium discoideum (DdPFK, EC2.7.1.11) following the procedure described in Santamaria et al., J. Biol. Chem., 277:1210-1216 (2002).

Site-directed mutagenesis. Point mutations were created according to the manufacture's protocol for the Scuptor In vitro Mutagenesis System, using the single-stranded DNA derived from the plasmid pMDDdPFK. This plasmid contained the full-length cDNA of DdPFK and was constructed by cloning the 2.7-kb Bam H1 fragment from pE3 into the M13 mp19 victor. The mutagenic primers used are described in the following table. Sequences of the mutagenic primers used in this study are listed in the following Table 2.

TABLE 2
Mutation Synthetic oligodeoxynucleotides (5′ to 3′)^{a}

| | | |
|---|---|---|
| L834A | TATTTATGCAGTAAATTGTGGATTAAC | (SEQ ID NO:1) |
| Δ 36 | GAgaattcTTAATTACAACCTTCGA | (SEQ ID NO:2) |
| Δ 26 | CTCgaattcTTAAATAGGTCTATCTTGTTCCG | (SEQ ID NO:3) |
| Δ 14 | CTCgaattcTTAAGAATAAGAGGTTGGAGAAG | (SEQ ID NO:4) |
| Δ 8 | GGgaattcTTATGGGTCAAAAGTTTTTTGAG | (SEQ ID NO:5) |
| Δ 4 | AGgaattcTTATGGATTAACATTTG | (SEQ ID NO:6) |
| Δ 2 | AGgaattcTTAAAATTGTGGATTAAC | (SEQ ID NO:7) |
| Δ 1 | AGgaattcTTATTAAGTAAATTGTGGATTAACATTTGG | (SEQ ID NO:8) |

α The introduced mutations are in bold face. EcoRI restriction site is in lowercase. Stop codons are underlined.

C-terminal deletion mutants were constructed by PCR employing mutagenic primers that generated the TAA stop codon at the desired position and an EcoRI site at the 3′ end of the mutated pfk gene. To obtain the D26 mutant, the 634-bp fragment of the 3′ end of DdPFK cDNA was isolated from the plasmid pE3 (27) by NheI-digestion, blunt-ending, and SpeI-digestion, and then cloned into pBluescript II SK⁺ that had been XbaI-digested, blunt-ended, and SpeI-digested yielding pMUT1, which was used as a template. The PCR product was NheI-EcoRI-digested and ligated with a HindIII-NheI fragment from pE3 (containing the remained 2002 bp of the 5′ end of DdPFK cDNA) and HindIII-EcoRI-digested pBluescript II SK⁺ to give pMUT2. The plasmid pMUT1 was used as a template to generate all other deletion mutants and their corresponding PCR products were cloned as NheIEcoRI fragments into pMUT2. All mutants were verified by DNA sequencing. For expression in yeast, mutant pfk genes were inserted as BamHI fragments downstream of the PFK2 promoter of Saccharomyces cerevisiae in the plasmid pJJH71.

Expression and Purification of Recombinant Enzymes. The plasmids containing either wild-type (Estevez and Heinisch, FEBS Lett. 374:100-104 (1995)) or mutant PFK cDNA are expressed in a S. cerevisiae strain HD 152-ID carrying deletions in both yeast PFK genes. Yeast transformants are grown in 1 liter of rich medium containing glucose to early stationary phase and recombinant enzymes are purified by 10% (w/v) polyethylene glycol (PEG) fractionation and chromatography on DE52 and blue Sepharose CL-6B as described previously (Estevez and Heinisch, FEBS Lett. 374:100-104 (1995)), except that PFK activity is eluted from the latter column with a 100-ml linear gradient of 0-1.5 M KCl in equilibration buffer. All final preparations of recombinant enzymes are judged to be homogeneous or not by SDS-PAGE analysis on 10% gels and Coomassie Blue staining.

FPLC Size-exclusion Chromatography. PFK samples dialyzed against 50 mM Na₂HPO₄, 100 mM KCl, 5 mM MgCl₂, 1 mM EDTA, pH 6.8, are applied to a Amersham Bioscience, Inc. Superose 6 HR 10/30 column equilibrated and eluted with the same buffer at a flow rate of 0.3 ml/min. Fractions of 0.11 ml are collected and tested for enzyme activity.

Other Methods. Protein level is determined by the method of Bradford. Permeabilization of yeast cells is carried out with a toluene/ethanol/Triton X-100 mixture as described in Aragon and Sanchez, Biochem. Biophys. Res. Commun. 131. 849-855 (1985). Mass spectrometry of recombinant enzymes is performed on a Bruker (Bremen, Germany) Reflex II matrix-assisted laser desorption ionization-time of flight mass spectrometer. ELISA analysis is performed in antibody capture mode using 1/2,000 dilution of polyclonal rabbit antibody against DdPFK and assaying peroxidase; samples containing no antigen are used as a blank. Prediction of secondary structure is obtained from the PHD server.

Stimulation Assay of DdPFK Phosphofructokinase and their mutants by Fructose 2,6 Pi. PFK activity is measured in an assay mixture that unless otherwise indicated contained 50 mM Hepes, 100 mM KCl, 5 mM MgC12, pH 7.2, 0.15 mM NADH, 1 mM MgATP, 1.2 units of aldolase, 10 units of triose-phosphate isomerase, 1 unit of glycerol-3-β dehydrogenase, and 5-10 ml of the purified enzyme in a total volume of 1 ml. After 5 min, the reaction is started by adding 1 mM Fru-6-P and is followed by measuring the absorbance change at 340 nm at 25° C.

When PFK activity is assayed in permeabilized cells, Glu-6-P is added to pyruvate kinase (1 unit) and lactate dehydrogenase (1 unit) is used as coupling enzymes in the presence of 0.2 mM P-enolpyruvate. Auxiliary enzymes are desalted by centrifugation and dialysis against 10 mM Hepes, pH 7.0, 20% (v/v) glycerol. One unit of activity is defined as the amount that catalyzes the conversion of 1 μmol/min of substrate at 25° C.

### Example 4

### Synthesis of 1-substituted-β-D-fructofuranose 2,6-bisphosphate (SFT-BP) (9)

A synthesis scheme of 1-substituted-β-D-fructofuranose 2,6-bisphosphate (SFT-BP) (9) is illustrated in FIG. 5.

Synthesis of 1,2-O-isopropylene-β-D-fructofuranose (1): A mixture of D-fructose (18 g, 100 mmol), 2-dimethoxypropene (33.52 ml, 350 mmol), and tin (II) chloride (107 mg, 0.56 mmol) suspended in dry 1,2-dimethoxyethane (1000 ml) was refluxed for 25 min. The reaction was terminated by the addition of pyridine (0.72 ml). The solvents were removed by rotary evaporation and the residue was purified with column chromatography (hexane/ethyl acetate=1:1 and then ethyl acetate only). Product: 6.6 g. Yield: 30%.

Synthesis of 1,2-O-isopropylene-6-O-(tert-butyldimethylsilyl)-β-D-fructofuranose (2): To the reaction solution of 1,2-isopropylene-β-D-fructofuranose 1 (9 g, 40.9 mmol), imidazole (3.4 g, 50 mmol) and DMF (80 mL) pre-cooled in an ice bath was added dropwise a solution of tert-butyldimethylsilyl chloride (6.48 g, 43 mmol) dissolved in DMF (20 mL) in a period of 30 min. After stirring for 10 h at 4° C., the solvent was removed by rotary evaporation at room temperature. The crude product was purified with column chromatography (hexane/ethyl acetate=1:4 and then 1:1). Ten and half (10.5) g of compound 2 was obtained. Yield: 77%.

Synthesis of 1,2-O-isopropylene-3,4-di-O-benzyl-6-O-(tert-butyldimethylsilyl)-β-D-fructofuranose (3): After A solution of 1,2-O-isopropylene-6-O-(tert-butyldimethylsilyl)-β-D-fructofuranose 2 (10.5 g, 31.42) dissolved in DMF (100 mL) was treated with sodium hydride (5.03 g, 60% in mineral oil, 125.68 mmol) for 2 h at room temperature, benzyl bromide (30 mL, 251.36 mmol) was added. The reaction mixture was stirred over night at room temperature and then 1 h at 60° C. After cooling down to room temperature, 10 mL of methanol was added. Solvents were removed by rotary evaporation at 70° C. and the residue was treated with ethyl acetate (100 mL). After the solid was removed with filtration, the filtrate was concentrated and purified with column chromatography (hexane/ethyl acetate=10:1 and then 5:1). Product 3: 6.5 g. Yield: 40%.

Synthesis of 3,4-di-O-benzyl-6-O-(tert-butyldimethylsilyl)-β-D-fructofuranose (4): To 1,2-O-isopropylene-3,4-di-O-benzyl-6-O-(tert-butyldimethylsilyl)-β-D-fructofuranose (3) (6.5 g, 12.65 mmol) was added a mixture of methylene chloride and trifluoroacetic acid (20 mL, 1:1). After stirring for 0.5 h at room temperature, the solvents was removed by rotary evaporation and then residue was purified with column chromatography (hexane/ethyl acetate=1:1 and then ethyl acetate only). Compound 4: 4.8 g. Yield: 80%.

Synthesis of 1-O-tosyl-3,4-di-O-benzyl-6-O-(tert-butyldimethylsilyl)-β-D-fructofuranose (5): 3,4-di-O-benzyl-6-O-(tert-butyldimethylsilyl)-β-D-fructofuranose 4 (4.0 g, 8.44 mmol) dissolved in pyridine (20 mL) was stirred with p-toluenesulfonyl chloride (7.82 g, 4.1 mmol) overnight at 4° C. After pyridine was removed by rotary evaporation, the residue was dissolved in chloroform (100 mL) and washed with water (2×100 mL), saturated sodium bicarbonated and then dried with anhydrous magnesium sulfate. The solvent was removed by rotary evaporation and the residue was purified with column chromatography (hexane/ethyl acetate=1:1, 1:2 and then 1:4). Compound 5: 3.8 g. Yield: 72%.

Synthesis of 1-O-tosyl-3,4-di-O-benzyl-β-D-fructofuranose (6): To a solution of 1-O-tosyl-3,4-di-O-benzyl-6-O-(tert-butyldimethylsilyl)-β-D-fructofuranose 5 (3.8 g, 6 mmol) and THF (50 mL) was added tetrabutylammonium fluoride (6 mL of 1 M TBAF in THF, 6 mmol). After stirring for 0.5 h at room temperature, THF was removed by rotary evaporation and the residue was purified with column chromatography (hexane/ethyl acetate=1:1, 1:2 and then 1:4). Compound 5: 2.72 g. Yield: 88%.

Synthesis of 1-O-tosyl-3,4-di-O-benzyl-β-D-fructofuranose 2,6-bis(dibenzyl phosphate) (7): To a solution of 1-O-tosyl-3,4-di-O-benzyl-β-D-fructofuranose (6) (2.6 g, 5 mmol) in THF (20 mL) was added 1,2,4-triazole (0.48 g, 7 mmol) and dibenzyl diethylphosphoramidite (1.9 g, 6 mmol) and the mixture was stirred for 24 h at room temperature. It was then cooled to −78° C., hydrogen peroxide (30%) was added in a single portion. The reaction mixture was allowed to warm to room temperature with stirring for 1.5 h. After removing of solvents, the residue was dissolved in ether (100 mL) and washed with 2N sodium hydrogensulfite (2×20 mL) and saturated sodium chloride (30 mL). The organic layer was dried with anhydrous magnesium sulfate and the solvent was removed by rotary evaporation. The product was purified with column chromatography (hexane/ethyl acetate=4:1, 2:1 and then 1:1). Compound 7: 2.5 g. Yield: 48%.

Synthesis of 1-substittuted-3,4-di-O-benzyl-β-D-fructofuranose 2,6-bis(dibenzyl phosphate) (8): Various 1-substittuted-3,4-di-O-benzyl-β-D-fructofuranose 2,6-bis(dibenzyl phosphate) (8) are synthesized by using the reaction of the leaving group tosyl of compound 7 with nucleophiles R₁-R₂-R₃. Here R₁ ═O, or N, or S etc.; R₂=alkyl group (C3 to C30); R₃=active ester, or SH, or NH2 etc.

Synthesis of 1-substittuted-β-D-fructofuranose 2,6-bisphosphate (SFI-BP) (9):

A solution of compound 8 (1 mmol) in methanol (12 mL) and ethyl acetate (3 mL) is shaken in a Parr hydrogenator over palladium hydroxide-on carbon (100 mg) for 10 h at 74 psi. The solid is removed by filtration and then the solvent is evaporated. The gummy product is characterized to test the presence or amount of compound 9.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

The claimed invention is:

1. A method for assaying an analyte in a sample comprising:
a) contacting a sample containing an analyte or suspected of containing an analyte with a specific binding reagent for said analyte in the presence of an allosteric phosphofructokinase and an analyte or analyte analog conjugated to a fructose-2,6-bisphosphate or fructose-1,6-bisphosphate compound, under conditions such that binding of said specific binding reagent to said analyte or analyte analog conjugated to said fructose-2,6-bisphosphate or fructose-1,6-bisphosphate compound prevents or reduces regulation of said allosteric phosphofructokinase by said fructose-2,6-bisphosphate or fructose-1,6-bisphosphate compound, and said analyte, if present in said sample, competes with said analyte or analyte analog conjugated to said fructose-2,6-bisphosphate or fructose-1,6-bisphosphate compound for binding with said specific binding reagent and reduces or prevents binding of said specific binding reagent to said analyte or analyte analog conjugated to said fructose-2,6-bisphosphate or fructose-1,6-bisphosphate compound, leading to increased regulation of said allosteric phosphofructokinase; and
b) determining the presence, absence and/or amount of said analyte in said sample by assessing activity of said allosteric phosphofructokinase.

2. The method of claim 1, wherein the sample is a biosample.

3. The method of claim 1, wherein the analyte is selected from the group consisting of a small molecule, a protein, a peptide, a hormone, a nucleic acid molecule, a fatty acid, and a saccharide.

4. The method of claim 1, wherein the analyte is thyroid hormone T3 or T4.

5. The method of claim 1, wherein the specific binding reagent is selected from the group consisting of a small molecule, a protein, a peptide, a hormone, a nucleic acid, a oligonucleotide, a fatty acid, a saccharide, and a polysaccharide.

6. The method of claim 1, wherein the specific binding reagent is an antibody or a soluble receptor.

7. The method of claim 1, wherein the allosteric phosphofructokinase is a native or a recombinant phosphofructokinase.

8. The method of claim 1, wherein the activity of the phosphofructokinase is assessed by measuring NAD⁺ consumed or NADH generated in the presence of D-fructose-6-phosphate, ATP, phosphate, and NAD⁺.

9. The method of claim 1, wherein the activity of the phosphofructokinase is assessed by measuring NAD⁺ consumed or NADH generated in the reactions catalyzed by fructose-bisphosphate aldolase and glyceraldehyde-3-phosphate dehydrogenase.

10. The method of claim 1, wherein the activity of the phosphofructokinase is assessed by measuring β-NADH consumed or β-NAD⁺ generated in the presence of D-fructose-6-phosphate, ATP, phospho(enol)pyruvate and b-NADH.

11. The method of claim 1, wherein the activity of the phosphofructokinase is assessed by measuring β-NADH consumed or β-NAD⁺ generated in the reactions catalyzed by pyruvate kinase and lactic acid (or lactate) dehydrogenase.

12. The method of claim 1, wherein the activity of the phosphofructokinase is assessed by measuring NAD⁺ consumed or NADH generated in the presence of D-fructose-6-phosphate, pyrophosphate, phosphate, and NAD⁺.

13. The method of claim 1, wherein the activity of the phosphofructokinase is assessed by measuring NADH consumed or NAD⁺ generated in the presence of D-fructose-6-phosphate, pyrophosphate, NADH, aldolase, triose phospphate isomerase and glycerol-3-phosphate dehydrogenase.

14. The method of claim 1, wherein the fructose-2,6-bisphosphate or fructose-1,6-bisphosphate compound is covalently conjugated to the analyte or analyte analog.

15. The method of claim 1, wherein the fructose-2,6-bisphosphate or fructose-1,6-bisphosphate compound is fructose-2,6-bisphosphate or fructose-1,6-bisphosphate.

16. The method of claim 1, wherein the fructose-2,6-bisphosphate compound has the following formula I: 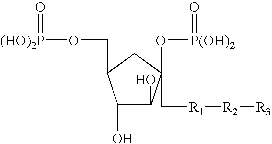
wherein
R₁ is O, N or S;
R₂ is a C₃ to C₃₀ alkyl group; and
R₃ is OH, SH, NH₂, COOH, CONH₂, or COOR₄, wherein R₄ is a C₁ to C₃₀ alkyl group.

17. A kit for assaying an analyte in a sample comprising:
a) a specific binding reagent for an analyte;
b) an allosteric phosphofructokinase;
c) the analyte or analyte analog conjugated to fructose-2,6-bisphosphate or fructose-1,6-bisphosphate compound; and
d) means for assessing activity of said allosteric phosphofructokinase.

18. The kit of claim 17, wherein the sample is a biosample.

19. The kit of claim 17, wherein the analyte is selected from the group consisting of a small molecule, a protein, a peptide, a hormone, a nucleic acid molecule, a fatty acid, and a saccharide.

20. The kit of claim 17, wherein the analyte is thyroid hormone T4.

21. The kit of claim 17, wherein the specific binding reagent is selected from the group consisting of a small molecule, a protein, a peptide, a hormone, a nucleic acid, a oligonucleotide, a fatty acid, a saccharide, and a polysaccharide.

22. The kit of claim 17, wherein the specific binding reagent is an antibody or a soluble receptor.

23. The kit of claim 17, wherein the allosteric phosphofructokinase is a native or a recombinant phosphofructokinase.

24. The kit of claim 17, wherein the means for assessing activity of the allosteric phosphofructokinase comprises D-fructose-6-phosphate, ATP, phosphate, and NAD⁺.

25. The kit of claim 17, wherein the means for assessing activity of the allosteric phosphofructokinase comprises fructose-bisphosphate aldolase and glyceraldehyde-3-phosphate dehydrogenase.

26. The kit of claim 17, wherein the means for assessing activity of the allosteric phosphofructokinase comprises D-fructose-6-phosphate, ATP, phospho(enol)pyruvate and b-NADH.

27. The kit of claim 17, wherein the means for assessing activity of the allosteric phosphofructokinase comprises pyruvate kinase and lactate dehydrogenase.

28. The kit of claim 17, wherein the means for assessing activity of the allosteric phosphofructokinase comprises D-fructose-6-phosphate, pyrophosphate, phosphate, and NAD⁺.

29. The kit of claim 17, wherein the means for assessing activity of the allosteric phosphofructokinase comprises D-fructose-6-phosphate, pyrophosphate, NADH, fructose-bisphosphate aldolase, triose phospphate isomerase and glycerol-3-phosphate dehydrogenase.

30. The kit of claim 17, wherein the fructose-2,6-bisphosphate or fructose-1,6-bisphosphate compound is covalently conjugated to the analyte or analyte analog.

31. The kit of claim 17, wherein the fructose-2,6-bisphosphate or fructose-1,6-bisphosphate compound is fructose-2,6-bisphosphate or fructose-1,6-bisphosphate.

32. The kit of claim 17, wherein the fructose-2,6-bisphosphate compound has the following formula I: 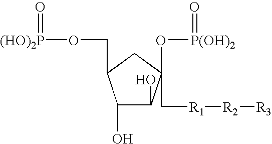
wherein
R₁ is O, N or S;
R₂ is a C₃ to C₃₀ alkyl group; and
R₃ is OH, SH, NH₂, COOH, CONH₂, or COOR₄, wherein R₄ is a C₁ to C₃₀ alkyl group.

33. A 1-substituted-β-D-fructofuranose 2,6-bisphosphate compound, or a salt thereof, having the following formula I: 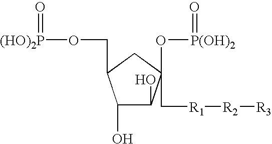
wherein
R₁ is O, N or S;
R₂ is a C₃ to C₃₀ alkyl group; and
R₃ is OH, SH, NH₂, COOH, CONH₂, or COOR₄, wherein R₄ is a C₁ to C₃₀ alkyl group.

34. An analyte, analyte analog or a specific binding partner for an analyte conjugated to the 1-substituted-β-D-fructofuranose 2,6-bisphosphate compound of claim 33.
